(19) United States
(12) Patent Application Publication
(10) Pub. No.: US 2026/0208171 A1
(43) Pub. Date: Jul. 23, 2026

(54) CATALYSTS AND METHOD FOR LIGHT HYDROCARBON SYNTHESIS USING RENEWABLE SYNGAS

(71) Applicant: ENERKEM INC., Montréal (CA)

(72) Inventors: Prashant KUMAR, Edmonton (CA); Samira ROSTOM, Nepean (CA); Hessam ZIAEI-AZAD, Edmonton (CA); Ce YANG, Clarksville, MD (US)

(21) Appl. No.: 19/141,135

(22) PCT Filed: Dec. 15, 2023
(86) PCT No.: PCT/CA2023/051673

§ 371 Date: Jun. 19, 2025

Related U.S. Application Data
(60) Provisional application No. 63/434,894, filed on Dec. 22, 2022.

(51) Int. Cl. B01J 29/072; B01J 35/00; C07C 1/04

(52) U.S. Cl. CPC B01J 29/072; B01J 35/19; C07C 1/044; C07C 2529/072

(57) ABSTRACT

There is provided a catalyst for the direct conversion of syngas into light olefins. The catalyst contains a mixture of a first catalytic component and a second catalytic component. The first catalytic component includes an oxide of a metal selected from the group consisting of copper, silver, iron, zinc, boron, magnesium, cobalt, aluminum, vanadium, nickel, yttrium, chromium, manganese, palladium, lanthanum, zirconium, and mixtures thereof. The second component includes a zeolite.

### CROSS-REFERENCE TO RELATED APPLICATION

The present application is claiming priority from U.S. Provisional Application No. 63/434,894 filed Dec. 22, 2023, the content of which is hereby incorporated by reference in its entirety.

### TECHNICAL FIELD

This disclosure relates to the field of light hydrocarbons, methods of making same and catalysts for making same.

### BACKGROUND OF THE ART

Light olefins such as ethylene, propylene, and butylene are considered the most widely used petrochemical feedstocks for use as chemical intermediates for production of solvents, polymers, plastics, fibers, and detergents. Light olefins are traditionally produced from naphtha, or natural gas (e.g. ethane, propane, butane). To transition the olefin industry to a more green and renewable space, the production methods and the raw material used to produce the light olefins must be diversified. Compared to conventional plastics production, a circular solution that eliminates new waste entering the ecosystem and that is less carbon intensive is desired. One of the circular solutions is chemical recycling via syngas. Syngas is a key platform for the utilization of renewable carbon feedstocks such as biomass, municipal solid waste (MSW) and non-recyclable plastic waste.

It would be desirable to leverage syngas from renewable sources for the production of light olefins in a more environmentally friendly manner.

### SUMMARY

In one aspect, there is provided a catalyst for the direct conversion of syngas into light olefins, the catalyst comprising a mixture of a first catalytic component and a second catalytic component, the first catalytic component comprising an oxide of a metal selected from the group consisting of copper, silver, iron, zinc, boron, magnesium, cobalt, aluminum, vanadium, nickel, yttrium, chromium, manganese, palladium, lanthanum, zirconium, and mixtures thereof, and the second component comprising a zeolite.

In some embodiments, the oxide of the metal of the first catalytic component comprises iron and an oxide of zinc, aluminum, manganese, copper, cobalt, and/or zirconium.

In some embodiments, the zeolite is KFI zeolite, mordenite (MOR), metal modified MOR, and metal modified KFI.

In some embodiments, the metal modified KFI is a mesoporous KFI zeolite containing an oxide of cerium.

In some embodiments, the first catalytic component is an iron-based catalyst that comprises zinc oxide, manganese oxide, or a combination thereof.

In some embodiments, the zeolite is a small or medium pore KFI type material.

In some embodiments, the catalyst comprises from 3 wt. % to 7 wt. % Fe.

In some embodiments, the catalyst comprises from 0.5 wt. % to 1.0 wt. % Zn.

In some embodiments, the catalyst comprises from 0.5 wt. % to 4.0 wt. % Ce.

In some embodiments, the catalyst comprises 53 to 56 wt. % of O, 5.5 to 7.5 wt. % of Al, 33 to 33.5 wt. % of Si, and 0 to 3.5 wt. % Fe.

In some embodiments, the catalyst comprises 55.04 wt. % O, 5.62 wt. % Al, 35.07 wt. % Si and 0.25 wt. % Fe.

In some embodiments, the catalyst comprises 53.16 wt. % O, 6.01 wt. % Al, 33.22 wt.

% Si, 2.98 wt. % Fe, 0.75 wt. % Na and 6.42 wt. % K.

In some embodiments, the catalyst comprises 55.67 wt. % O, 7.37 wt. % Al, 33.66 wt.

% Si, 2.82 wt. % Fe, and 1.31 wt. % K.

In some embodiments, the catalyst comprises 55.53 wt. % O, 7.29 wt. % Al, 33.44 wt.

% Si, 2.85 wt. % Fe, and 0.13 wt. % K.

In one aspect there is provided a method of producing the catalyst of the present disclosure, the method comprising mixing the first catalytic component with the second catalytic component to obtain a solid powder mixture, and heating the solid powder mixture at a first temperature of from 65° C. to 150° C.

In some embodiments, the method further comprises heating at a second temperature of from 500° C. to 600° C. after the first temperature.

In some embodiments, the temperature is increased from the first to the second at a rate of at least 2° C./minute.

In some embodiments, the heating is performed at atmosphere with air.

In one aspect there is provided a process for producing light olefins from a syngas mixture comprising H₂, CO and CO₂ comprising contacting the syngas with the catalyst as defined in any one of claims 1 to 9 in a converter unit to form a product comprising C₁ to C₄ light olefins.

In some embodiments, the syngas is obtained from a carbonaceous material that comprises a biomass, a plastic, an organic compound, industrial wastes, recycling facilities rejects, automobile fluff, municipal solid waste, construction and demolition debris, refuse derived fuel (RDF), solid recovered fuel, used wood utility poles, wood railroad ties, wood waste recovered form forestry, tire, synthetic textile, carpet, synthetic rubber, materials of fossil fuel origin, expanded or any combination thereof.

In some embodiments, the syngas converter unit is operated at a temperature of between 200° C. and 400° C.

In some embodiments, the syngas converter has a pressure of from 200 psig to 1000 psig.

In some embodiments, the syngas is provided to the syngas converter unit at a space velocity of from 1000 to 5000 ml/h/g cat.

In some embodiments, the catalyst has a CO conversion in a range from 60 mol % to 95 mol %.

In some embodiments, the catalyst has a CO₂ selectivity in a range of 10 mol % to 40 mol %.

In some embodiments, the catalyst has a CH₄ selectivity has a range of 7 mol % to 45 mol %.

In some embodiments, the catalyst has C₂ to C₄ olefines selectivity in a range of 10 mol % to 25 mol %.

In some embodiments, the catalyst has C₂ to C₄ paraffins selectivity in a range of 4 mol % to 40 mol %.

In some embodiments, the catalyst has a C₅⁺ selectivity in a range of 3 mol % to 25 mol %.

In some embodiments, the catalyst has a selectivity range of 60 mol % to 98 mol % for C1 to C4 hydrocarbons on a CO₂ free basis

In some embodiments, a ratio of hydrogen to carbon monoxide in the synthesis gas is from 0.5:1 to 5:1.

In some embodiments, the syngas is produced from a carbonaceous gas component comprises a mixture of hydrogen and carbon monoxide and carbon dioxide with CO to CO₂ ratio between 1:1 and 3:1.

In some embodiments, an excess CO₂ produced through the process is repurposed to produce more syngas through dry reforming or steam-aided dry reforming.

In some embodiments, the syngas converter unit is a fixed bed reactor.

Many further features and combinations thereof concerning the present improvements will appear to those skilled in the art following a reading of the instant disclosure.

### DESCRIPTION OF THE DRAWINGS

FIG. 1 is a schematic illustration of a waste valorization that includes plastic to syngas through gasification units followed by its single-step conversion to olefin rich stream and a separation train according to one embodiment of the present disclosure.

FIG. 2 is a graph showing a representative example of the X-Ray diffractogram of the zeolites used in the syngas conversion process.

FIG. 3 is a process flow diagram (PFD) of the testing apparatus used in the Example section.

FIG. 4 is a graph showing a conversion and selectivity comparison of the catalysts tested (effect of synthesis techniques adopted).

FIG. 5 is a graph showing a conversion and selectivity comparison (effect of Zn addition).

FIG. 6 is a graph showing a conversion and selectivity comparison (effect of higher iron concentration).

FIG. 7 is a graph showing a conversion and selectivity comparison (effect of zeolite presence in the formulation)

FIG. 8 is a graph showing a conversion and selectivity comparison (effect of higher CO₂ concentration).

FIG. 9 is a graph showing a conversion and selectivity comparison (effect of meso-porosity).

FIG. 10 is a graph showing the CO conversion profiles of the representative catalysts as a function of time-on-stream (TOS).

### DETAILED DESCRIPTION

The present disclosure provides a direct light hydrocarbon formation catalyst, a method of making the catalyst, and a process for using the catalyst to make an olefin product. The light hydrocarbons obtained are particularly high in ethylene and propylene content.

The present disclosure relates to the Fischer-Tropsch synthesis of light hydrocarbons by renewable syngas stream of appropriate ratio (e.g. H₂/CO=1 to 3) using a catalyst. In some embodiments, the synthesis gas has a ratio of hydrogen to carbon monoxide of from 0.5:1 to 5:1 or 0.5:1 to 4:1. Synthesis gas, also called syngas, is a fuel gas mixture comprising primarily of carbon monoxide (CO), carbon dioxide (CO₂) and hydrogen (H₂). Syngas can be produced from many sources, including biomass, or other carbonaceous materials, by reaction with steam (steam reforming), carbon dioxide (dry reforming), air (partial oxidation), oxygen (partial oxidation) or any mixture thereof.

In general, urban heterogeneous wastes are materials which are obtained from non-recycled plastics, municipal solid, and its derivatives such as refuse derived fuel, forestry waste etc. Gasification of such waste materials are known to those skilled in the art. For example, in a non-limiting embodiment, the biomass may be gasified in a gasifier which includes a fluidized bed section and a reforming, or freeboard section. Examples of such gasifiers to produce clean syngas are disclosed in published patents such as U.S. Pat. Nos. 8,080,693, 8,436,215, 8,137,655, 8,192,647, and 8,636,923 which are incorporated herein by reference in their entirety. The carbonaceous materials encompassed herein may also be any type of coal and derivative such as pet coke, petroleum products and by-products, waste oil, oily fuel, hydrocarbons, and tar, although renewable sources are preferred.

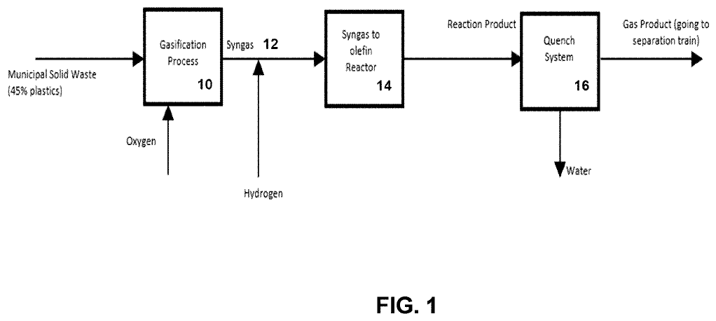

Converting carbonaceous materials and waste into synthesis gas can be achieved with gasification techniques. Syngas may be produced by gasifying carbonaceous feedstock (FIG. 1). The gasification 10 provides a crude syngas 12 which includes impurities such as ammonia (NH₃), sulfur (as hydrogen sulfide (H₂S) and carbonyl sulfide (COS)), chlorine (as HCl), volatile metals, aromatic tars (NBTX; naphthalene, benzene, toluene, and xylene), tars, fines ashes (in the form of particles containing metals and metal salts), bed material, and char (solid particulates typically above 0.001 mm and containing metals, salts and mostly carbon). Such impurities, however, limit the ability of the syngas to be used as a fuel or to be employed in the synthesis of other useful materials without a cleaning process.

Syngas 12 is used as a feedstock to form a composition comprising a major amount of light range hydrocarbons. In some embodiments, the synthesis gas feed (including any recycle syngas recovered from the process itself, as well as fresh syngas) has a molar ratio of hydrogen (H₂) to carbon oxides (CO+CO₂) in the range of from about 0.5:1 to about 20:1, preferably in the range of from about 1:1 to about 10:1. In another embodiment, the synthesis gas has a molar ratio of hydrogen (H₂) to carbon monoxide (CO) of at least 2:1. Carbon dioxide is, optionally, present in an amount of not greater than 50% by weight, based on total weight of the synthesis gas, and preferably less than 35% by weight, more preferably less than 25% by weight.

The stoichiometric molar ratio of the syngas, defined as the molar ratio of (H₂+CO₂)/(CO+CO₂), should be sufficiently high so as to maintain a high selectivity to C2-C4 light hydrocarbons. In one embodiment, the synthesis gas used as feed for producing the desirable product profiles has a stoichiometric molar ratio of from about 1.0:1 to about 2.7:1, more preferably from about 1.5:1 to about 2.5:1, more preferably a stoichiometric molar ratio of from about 1.75:1 to about 2.5:1. A H₂/CO molar ratio is of 2 is preferred and a CO/CO₂ molar ratio of 3 is also preferred.

The synthesis of olefins from syngas is preferably performed in a syngas converter unit 14 (or syngas to olefin reactor), such as a fixed bed reactor preferably having a plug-flow behaviour. Afterwards, the reaction product is quenched 16 and the gas product is then further processed.

The direct conversion of syngas to light hydrocarbon can be accomplished over a wide range of temperatures. Lower to mid-temperature ranges were found to be preferred. In one embodiment, the syngas is contacted with the catalysts at a temperature in the range of from about 150° C. to about 550° C., preferably in a range of from about 175° C. to about 450° C., from about 200° C. to about 500° C., more preferably in a range of from about 200° C. to about 400° C. The syngas can also be converted to an intermediate composition over a wide range of pressures. In one embodiment, the syngas is contacted with the catalyst at a pressure in the range of from about 15 atmospheres to about 125 atmospheres, preferably in a range of from about 20 atmospheres to about 100 atmospheres, more preferably in a range of from about 25 atmospheres to about 75 atmospheres. Gas hourly space velocities in converting the syngas to alcohol product can vary depending upon the type of reactor that is used. In one embodiment, gas hourly space velocity of flow of gas through the catalyst bed is in the range of from about 50 hr⁻¹ to about 5,000 hr⁻¹. Preferably, gas hourly space velocity of flow of gas through the catalyst bed is in the range of from about 250 hr⁻¹ to about 3,000 hr⁻¹, from about 300 hr⁻¹ to about 3,000 hr⁻¹, or more preferably from about 500 hr⁻¹ to about 2,000 hr⁻¹. The catalytic conversion can be accomplished in a fixed-bed reactor in a temperature range from 300° C. to no more than 400° C. and a pressure range of 300 psig to 400 psig or from 100 psig to 900 psig. In some embodiments, the syngas is provided to the syngas converter unit at a space velocity of from 1000 to 5000 ml/h/g cat. The conditions provided herein are reaction conditions that favor the production of C2-C4 olefins and are selective for such products.

The catalyst of the present disclosure used for the conversion of syngas to light olefins comprises or consists of a mixture of a first catalytic component and a second catalytic component. The first catalytic component comprises an oxide of a metal selected from the group consisting of copper, silver, iron, zinc, boron, magnesium, cobalt, aluminum, vanadium, nickel, yttrium, chromium, manganese, palladium, lanthanum, zirconium, and mixtures thereof. The second component comprises or consists of a zeolite (aluminosilicate). The zeolite is preferably a KFI zeolite, a mordenite (MOR), a metal modified MOR, and/or a metal modified KFI. One example of a KFI is KFI is a mesoporous KFI zeolite containing an oxide of cerium. The pore size of the KFI material can be a small pore KFI defined as having pores in the range of 0.30-0.45 nm, a medium pore size KFI having pores ranging from 0.45-0.60 nm or a large pore size KFI having pores in the range of from 0.60-0.80 nm. In some embodiments, the KFI zeolite is a small to medium pore size zeolite (i.e. pores of 0.30-0.60 nm).

In some embodiments, the catalyst comprises from 3 wt. % to 7 wt. %, 3.5 wt. % to 6.5 wt. % or 4 wt. % to 6 wt. % Fe with respect to the total weight of the catalyst. In some embodiments, the catalyst comprises 0.5 wt. % to 1.0 wt. % Zn with respect to the total weight of the catalyst. In some embodiments, the catalyst comprises 0.5 wt. % to 4.0 wt. % Ce with respect to the total weight of the catalyst. In some embodiments, the catalyst comprises 52 to 57 wt. % of O, 5 to 8 wt. % of Al, 31 to 35 wt. % of Si, and 0 to 4 wt. % Fe. In some embodiments, the catalyst comprises 53 to 56 wt. % of O, 5.5 to 7.5 wt. % of Al, 33 to 33.5 wt. % of Si, and 0 to 3.5 wt. % Fe. The catalyst can optionally comprise up to 1 wt. % Na and up to 7 wt. % K.

The catalyst of the present disclosure can be produced by a method comprising mixing the first catalytic component with the second catalytic component to obtain a solid powder mixture, and heating the solid powder mixture at a first temperature of from 65° C. to 150° C. In some embodiments, the first temperature is maintained for 2±20% h, 2±10% h, 2±5% h or for 2 h. The method optionally comprises a further heating step at a second temperature of from 500° C. to 600° C. after the first temperature. In some embodiments, the second temperature is maintained for 3±20% h, 3±10% h, 3±5% h or for 3 h. In some embodiments, the temperature is increased from the first to the second at a rate of at least 2° C./minute.

The catalysts of the present disclosure advantageously achieve a selectivity for C2-C4 olefins as opposed to C5+ olefins or other paraffin products. The product profiles obtained are controllable with highest selectivity to lighter hydrocarbon such as C2 to C4 with less than 2 mol % of C5+ hydrocarbon. The formation of CO₂ and methane as other by-products of the conversion can also be controlled by the judicious choice of catalyst from the same family and under optimized process condition. In some embodiments, the catalyst has a CO conversion in a range from 60 mol % to 95 mol %. In some embodiments, the catalyst has a CO₂ selectivity in a range of 10 mol % to 40 mol %. In some embodiments, the catalyst has a CH₄ selectivity has a range of 7 mol % to 45 mol %. In some embodiments, the catalyst has C2 to C4 olefines selectivity in a range of 10 mol % to 25 mol %. In some embodiments, the catalyst has C2 to C4 paraffins selectivity in a range of 4 mol % to 40 mol %. In some embodiments, the catalyst has a C5⁺ selectivity in a range of 3 mol % to 25 mol %. In some embodiments, the catalyst has a selectivity range of 60 mol % to 98 mol % for C1 to C4 hydrocarbons on a CO₂ free basis.

The present disclosure also provides an integrated method of hydrocarbon synthesis from waste-plastic-derived-syngas through chemical recycling via gasification. Specifically, the disclosure relates to converting such waste materials to a composition of low boiling range hydrocarbons that are amenable to be utilized as sources for renewable plastic manufacturing. More specifically the present disclosure also relates to a system design that include a CO₂ converter through dry reforming as a downstream standalone reactor to produce additional syngas. More particularly, the invention relates to the composition, manufacture, and use of new coke-resistant catalysts with extended catalytic activity for producing lighter hydrocarbons (C2-C4) from sustainable resources.

Syngas is a key platform for the utilization of renewable carbon feedstocks such as biomass, municipal solid waste (MSW) and non-recyclable plastic waste. The catalytic transformation of syngas obtained from such waste, into value-added products with an emphasis on selective formations of lower range (C2-C4) hydrocarbons which are considered key building-block for chemicals such as plastic precursors would provide a path forward as drop-in alternatives. The approach to chemical recycling using waste plastic as a source of syngas production followed by a downstream conversion with appropriate selectivity would allow a renewable and sustainable pathway to plastic (polyethylene) production. A technology for preparing light hydrocarbon using syngas can widen the source of the raw material and will provide an alternative solution for a steam cracking technology.

The one-step direct preparation of the light olefins using the syngas is a process of directly preparing the light hydrocarbons with the number of carbon atoms less than or equal to 4 through Fischer-Tropsch synthesis (FTS) reaction of carbon monoxide and hydrogen under the action of the catalyst. This process simplifies the process integration with an existing asset and reduces the investment on methanol to olefins (MTO) route. FTS is a technology that allows the formation of predominantly straight-chain hydrocarbons, which can be paraffins from CH₄ to waxes (CₙH₂ₙ₊₂ with n from 1 to over 100), olefins from ethylene to much longer molecules (CₙH₂ₙ, with n=2), and to a lesser extent oxygenated products such as alcohols. It produces as main by-products water and/or carbon dioxide, that is, due to the water-gas shift (WGS) reaction. Being a highly exothermic reaction, it generates large amounts of heat. The process is represented by the simplified reaction equations:

$$\begin{matrix}FTS : CO + 2 {H}_{2} \to - C {H}_{2} - + {H}_{2} O & - 165 kj / mol\end{matrix} \qquad [ 1 ]$$

$$\begin{matrix}WGS : CO + {H}_{2} O \to {H}_{2} + {CO}_{2} & - 42 kj / mol\end{matrix} \qquad [ 2 ]$$

FTS is a surface-catalyzed polymerization process where the hydrogenation of adsorbed CO on a catalytic surface produce hydrocarbons with a broad range of chain lengths and functionality. Total product yield decreases exponentially with chain length, forming a so-called Anderson-Shultz-Flory (ASF) distribution. A Schulz-Flory distribution is a distribution of compounds that is expressed by the Schulz-Flory “alpha” (a) value. The higher the a number, as it approaches 1.0, the more selective a process is for producing wax molecules. One of the objectives of this disclosure is to shift the ASF distribution to a narrower range resulting into C2-C4 hydrocarbons selectively. The present disclosure provides a catalyst formulation and a method for preparing such in order to produce light hydrocarbon using direct conversion of syngas.

There exists several processes and methods of producing and treating synthesis gas in which a biomass-rich material is gasified in a gasifier containing a fluidized bed to produce a crude synthesis gas product. The crude synthesis gas then is quenched, scrubbed, and then subjected to at least one adsorption step to provide a clean synthesis gas. The clean synthesis gas then may be reformed catalytically to provide a synthesis gas with a desired H₂:CO ratio, and/or may be employed in the synthesis of desired chemicals (for example as described U.S. Pat. Nos. 8,137,655 and 8,192,647 which are incorporated herein by reference in their entirety). Examples of such gasification processes to generate such syngas are described in U.S. Pat. Nos. 8,137,655, 8,192,645, 8,436,215, 8,636,923 and 8,080,693, the contents of which are incorporated herein by reference in their entirety. If the waste material to be gasified is majorly plastic waste (for an advance recycling approach), the maximum utilization of such carbon present in the syngas through downstream catalytic conversion becomes particularly important.

Another aspect of the present disclosure relates to a method of synthesis of microporous matrix with zeolite like pores since the cavities present in the zeolites are crucial for activity and selectivity to the desirable range of lighter hydrocarbons including olefins. It was found that KFI type small pore zeolite is a preferred zeolite. In general, the KFI zeolites are composed of K₁₈Sr [Al₂₀Si₇₆O₁₉₂]·7H₂O (18 crown-6) intersecting with 8-membered rings (MR) with pore sizes of 3.9×3.9 Å. In addition, the tunability of this zeolite towards hierarchical structure is found to be very facile that not only provide stability but a tunable product selectivity. A hydrothermal synthesis procedure has been developed that include multiple steps of mixing together first and second component of dissolved liquid to a solid to form a slurry. The first and second component could be the oxides of alkali metals chosen from sodium and potassium in the form of nitrate salts. The solids are chosen from a commercial zeolite in the form of Faujasite type zeolite of appropriate Si/Al₂ ratio in order to affect the inter-zeolite transformation. The slurry is hydrothermally treated under autogenous pressure in an autoclave to generate catalyst samples. In a few other examples the oxides of metal of interest are added to the zeolite using wet impregnation or solid-solid ion exchange process. In all examples, the KFI structure of the zeolite were used as a carrier of the metals of interest.

In some embodiments, oxides of (ZnO, MnO), metals (Cu) and alkali (K, Na) oxides or carbonates are added to Fe oxide precursors as promoters to improve their structural integrity or catalytic properties. For example, ZnO, Cu, and K compounds can increase FTS rates on precipitated Fe₂O₃ precursors. Potassium promotes CO chemisorption and inhibits H₂ chemisorption, which in turn leads to lower FTS rates, higher product molecular weight, and greater olefin content. Cu, when present along with K, increases FTS rates without detectable changes in selectivity. Cu and K compounds have been reported to increase the activity for water-gas shift (WGS), a reaction that occurs concurrently with FTS on many Fe-based catalysts. Cu oxides in intimate mixtures with a Fe—Zn—K precursor matrix increase the rate of reduction and carburization of the Fe oxide component in these precursors, and leads to the formation of smaller FeCx crystallites, to greater active site densities, and to higher FTS rates. Hence, Cu does not act as chemical promoters, but instead provide a better dispersion of the active phase and a greater availability of active sites.

In further embodiments, the KFI can further be treated in order to improve the intra-crystalline diffusion limitation in the narrow micro-pores, since it may restrict the performance of zeolite in adsorption and desorption processes, central to catalytic conversion. Limitations in the diffusion not only reduce the catalytic performance but also affect the selectivity and durability of the catalyst.

In some embodiments, a complementary strategy to tackle diffusion limitations in zeolites was performed by incorporating the mesoporosity in the materials since mesopores have a desired pore size domain for improved mass transport as well as the well-defined morphology with uniform size (spheres in micro-size range) influences the rapid adsorption and desorption of the molecules. The combination of properties from different types of porosities could potentially enhance the overall mass transport of reagents and products to and from the catalytically active sites. It is demonstrated herein below that the combination of mesoporous and microporous pores in the zeolitic structures have exhibited high catalyst stability compared to microporous zeolite only.

In some embodiments, the catalyst is characterized in that one or more of hierarchical pores are in the form of Al₂O₃, SiO₂, Fe₂O₃, ZnO, K₂O, and Na₂O in the catalyst having a specific surface area in the range of 190-400 m²/g and pore volume in the range of 0.25-0.80 ml/g.

The dual-function composite catalyst can be used for preparing light olefins using one-step direct conversion of syngas, where the sum of the selectivity of ethylene and propylene reaches 40-60%; the sum of the selectivity of the light olefins comprising ethylene, propylene, and butylene and other C5 plus can reach 50-90%, while the selectivity of a methane byproduct is less than 15%.

Advantages of the catalysts, methods and processes described herein include:
preparation of the light olefins through one-step direct conversion of syngas,
the hierarchical pore carrier dispersed zeolite is beneficial to the mass transfer of the intermediate and the product, thereby greatly reducing the influence of side reactions such as hydrogenation and maintaining high selectivity of the light olefins while increasing the conversion rate, and
the catalyst has a simple preparation process that only require mild conditions.

Moreover, the process described herein can achieve an extremely high product yield and selectivity, with the selectivity for C2-C4 light olefins reaching 50-90% and especially can still maintain high alkene/alkane ratio after increasing the conversion rate. Meanwhile, the selectivity of the methane byproduct can be as low as <15%, and the catalyst has long service life which is longer than 700 hours.

### Example

A fixed-bed reactor with a plug-flow behaviour was constructed to evaluate catalysts. The reactor system also included a product analysis unit and liquid collection system. The reactor included a packed bed tubular reactor housed in a furnace with a single heating zone. The reactor tube was made from SS316 stainless-steel (Swagelok) which had an outer diameter of 0.5 inches, an internal diameter of about 0.4 inches, and a length of about twenty-two inches. The reactor was heated using a WATLOW heater equipped with a temperature limit controller. The thermocouple (K-type) having an outer diameter of 0.125 inches was inserted axially through the center of the reactor, which was used to measure and control the temperature within the catalyst bed of approximately 50 mm height. The particle size of the catalyst used were in the range of 0.71 mm to 0.5 mm. No diluents of any kind were used to prepare the catalysts prior to catalytic testing. The catalyst was housed on top of glass beads (Fischer™ Scientific, 5 mm size, 30 g) spaced by glass wool. Pure α-Al₂O₃ (Sasol™, ten gram) beads (0.5 mm diameter) calcined at 1100° C. were used on either end of the reactor tube before and after the catalyst bed and spaced by the glass wool. In total, the entire length of the reactor tube was filled up (approximately twenty inches) with inert materials to minimize the temperature gradient.

Further, in order to approach plug flow conditions and minimize back mixing and channeling, certain operating criteria such as the ratio of catalyst bed length to catalyst particle size (L/Dₚ) was maintained at more than fifty and the ratio of the inside diameter of the reactor to catalyst particle size (D/Dₚ) was maintained at more than 10. Prior to each experimental run for catalyst evaluation, the catalyst was activated by in situ reduction at 380-450° C. for 2-15 hours by flowing 10% H₂ in Ar (Linde™) using a mass flow controller (Bronkhorst™) at atmospheric pressure. The catalyst test was accomplished at temperature ranging from 350° C. to 380° C. Pressure was also varied from 300 to 400 psig. A premixed gas mixture (H₂/CO volume ratio two with 10% CO₂ by volume (Linde) was used as a feed. The gas hourly space velocity (GHSV) dictated the volume of gas flow rate depending on the volume of catalyst used in the experiment. Typically, the catalyst amount used was 2.0 grams at a given flow rate. GHSV was defined as volumetric flow of the reactor feed gas divided by the volume of the catalyst bed. The GHSV in mL/h/g_{cat} was calculated as

$$GHSV = \frac{V o l u m etric flow rate ( or feed flow rate )}{mass of the catalyst}$$

The feed and product gases were analyzed with an on-line gas chromatograph (7890B, Agilent™ Technologies). The GC was equipped with three detectors. The front flame ionization detector (FID) detected hydrocarbons from C1 to C9 and also separated ethane, ethylene, propane, propylene, butane, and butylene using an alumina plot column. The heavier hydrocarbons like aromatics (benzene, toluene, ethylbenzene, p-xylene, o-xylene, m-xylene), oxygenates (methanol, ethanol, and acetones etc.) were detected on another FID which used a CP Wax57 column. The permanent gases (H₂, O₂/Ar, N₂, CH₄, CO, CO₂) were detected on a TCD (thermal conductivity detector) and separated on a Haysep and molecular sieve column.

A chilled water condenser (Lauda™ chiller, operating at 5° C.) was located after the reactor to collect higher hydrocarbon and water condensates. The total gas volume after the reaction was calculated based on Ar that was used as an internal standard in the feed mixture. The conversion of CO and selectivity of CO₂ and C₂-C₄ olefins were calculated as described above.

The selectivity of ethylene only (S_{C}_{₂}_{H}_{₄}(%)), paraffins (S_{C}_{₂}_{-C}_{₄}₋(%)), C₅₊ (S_{C}_{₅}₊ (%)) and methane (S_{CH}_{₄}(%)) were calculated as follows:

$$\begin{matrix}{S}_{{C}_{2} {H}_{4}} ( \% ) = \frac{{n}_{{C}_{2} {H}_{4}}}{{n}_{CO} , in - {n}_{CO} , out} \times 100 \\ {S}_{{C}_{2} - {C}_{4} -} ( \% ) = \frac{{n}_{{C}_{2} {H}_{6}} + {n}_{{C}_{3} {H}_{8}} + {n}_{{C}_{4} {H}_{10}}}{{n}_{CO} , in - {n}_{CO} , out} \times 100 \\ {S}_{{C}_{5} +} ( \% ) = \frac{{n}_{{C}_{5} +}}{{n}_{CO} , in - {n}_{CO} out} \times 100 \\ {S}_{C {H}_{4}} ( \% ) = \frac{{n}_{C {H}_{4}}}{{n}_{CO} , in - {n}_{CO} , out} \times 100 \\ {Y}_{{C}_{2} =} ( mol \% ) = {S}_{{C}_{2} =} \times {X}_{CO} \div 100 \\ {Y}_{{C}_{2} - {C}_{4} =} ( mol \% ) = {S}_{{C}_{2} - {C}_{4}} \times {X}_{CO} \div 100\end{matrix}$$

n_{CO}, in is the moles of CO input. n_{CO}, out is the moles of CO output. n_{C}_{₂}_{H}_{₄} is the moles of C₂H₄ output while n_{C}_{₂}_{H}_{₆} is the moles of C₂H₆ output while n_{C}_{₃}_{H}_{₈} is the moles of C₃H₈ output while n_{C}_{₄}_{H}_{₁₀} is the moles of C₄H₁₀ output while, n_{CH}, is the moles of CH₄ output. Again the n_{C}_{₅₊} is the moles of C₅₊ output.

The syngas flow was controlled using a mass flow meter to get a certain composition of gas mixture. This was then fed to the reactor via a three-way valve. The valve allows to switch the feed to bypass while needed. The back pressure regulator (BPR) allows to control the reactor pressure. For all reporting data, the carbon balances were higher than 95%, and the selectivity were normalized to one hundred. The gas composition of the syngas getting into the reactor was as follows: 60% by volume H₂, 30% by volume CO and 10% by volume CO₂ having a GHSV of 1500 mL/h/g_{cat}, for the overall gas feed.

A co-precipitation method was used to prepare iron or modified iron catalysts with different structural promoters. For Zn-modified iron catalyst (Fe/Zn molar ratio=1/1), an aqueous solution containing Fe(NO₃)₃ (Sigma-Aldrich™, 98+%) and Zn(NO₃)₂ (Sigma-Aldrich™, 98%) was used as precursor, and 2 (M) Na₂CO₃ (Sigma-Aldrich™, 99.5+%) solution was used as precipitant. Under stirring, the precursor solution was added into a beaker containing 20 mL deionized water at 80° C. The Na₂CO₃ solution was simultaneously added to maintain the mixtures at pH 9. Then, the precipitate was aged for 4 h at room temperature and washed with certain amount of deionized water. Finally, the precipitate was dried at 120° C. overnight and subsequently calcined at 350° C. for 4 h in air. The sample was designated as X and used for the catalytic experiment as per the procedure vide supra.

A commercial sample of Na-MOR (Zeolyst CBV-10) was converted to NH₄-MOR through three consecutive aqueous ions exchange reactions. Using a 1-L round bottom flasks (RBFs) 10 g of Na-MOR and 500 mL of 1 M ammonium chloride (NH₄Cl) solution were mixed together. The contents of the RBFs were stirred at 80° C. for 3 hrs. Each solution was then filtered into their own respective Buchner funnel containing three qualitative filter papers. Each filter cake was washed with 500 mL of distilled water and dried in an oven at 90° C. overnight. The recovered amounts were close to eighty percent of the original charge. The procedure was repeated three times in order to complete three exchange to remove sodium and to convert the Na-MOR to NH₄-MOR. The NH₄-MOR was converted to H-MOR through calcination in air. The NH₄-MOR was loaded into a ceramic bowl and calcined using the muffle furnace. The furnace was ramped to 500° C. within 1 hour and held at 500° C. for 6 hours.

The H-MOR was impregnated with pyridine using a vacuum distillation setup. H-MOR was placed in a Kontes flask and evacuated to −30 mmHg at 210° C. for 4 hrs in a vacuum oven. The sample continued to be evacuated overnight and the oven was cooled back down to room temperature. The H-MOR Kontes was then connected to a vacuum distillation arm on a Schlenk line, with a second Kontes containing pyridine over molecular sieves connected at the other end of the arm. The H-MOR was further vacuum dried (<100 mTorr) as the pyridine Kontes was degassed using the freeze-pump-thaw procedure (repeated 3 times). The vacuum distillation arm was isolated under static vacuum, and the pyridine was allowed to thaw. The pyridine vapor was then transferred to the H-MOR Kontes, such that the entire sample was submerged in liquid. The H-MOR was submerged in pyridine for 30 minutes. Use of water heating bath under the pyridine Kontes and a cold bath under the H-MOR Kontes facilitated the transfer. Excess pyridine was transferred back to the pyridine Kontes by use of a heating bath under the H-MOR Kontes. The final consistency of the H-MOR powder was free-flowing in small granular clumps. The H-MOR Kontes was sealed under vacuum and transferred to a glove box for the sample to equilibrate overnight, remaining sealed and under reduced atmosphere. The sample was removed from the glovebox and transferred into a quartz boat. The boat was loaded into the split tube and purged with purified nitrogen for one hour with a flow rate of 85 sccm. The flow rate was reduced to 30 sccm and the sample was calcined under purified nitrogen at 500° C. for 4 hours with a 1-h ramp. There was 3.55 g of catalyst recovered after calcination and the material was an off-white color. The Py-MOR powder was pressed with 12 metric tons using a 1-inch die set, crushed using a mortar and pestle, then sieved to 500-710 μm.

CrZnAl oxide was synthesized according to a co-precipitation reaction as described in Jiao et al. ((2016). Science 351 (6277): 1065) and Jiao et al. ((2018). Angewandte Chemie International Edition 57 (17): 4692-4696). Zn(NO₃)₂·6H₂O (29.12 g, 97.89 mmol), Cr(NO₃)₂·9H₂O (11.2 g, 27.99 mmol), and Al(NO₃)₃·9H₂O (10.5, 27.99 mmol) were dissolved in 200 ml of distilled water in a 500-mL RBF. The solution was heated and stirred in a 70° C. oil bath. To the mixture was added dropwise 84 mL of 1 M (NH₄)₂CO₃ solution. The reaction was allowed to stir for 3 hours at 70° C. The solution was then filtered into a Buchner funnel containing three qualitative filter papers. The filter cake was washed with 2 L of distilled water and dried in an oven at 90° C. overnight. There was 8.56 g of material recovered, which had a light grey-blue color. The material was loaded into a ceramic bowl and calcined in the muffle furnace. The furnace was ramped to 500° C. within 30 minutes and held at 500° C. for 1 hour. There was 4.58 g of dark grey powder recovered after calcination. The Cr—Zn—Al oxide powder was pressed with 12 metric tons using a 1-inch die set, crushed using a mortar and pestle, then sieved to 500-710 μm. The Py-MOR pressed material and the Cr—Zn—Al oxide pressed material were combined in a 1:1 ratio by weight in a bottle. The bottle was shaken to obtain a homogeneous distribution of the catalyst designated as Cr—Zn—Al-Pyridine modified-MOR (or “Y”) and used for the catalytic experiment as per the procedure vide supra.

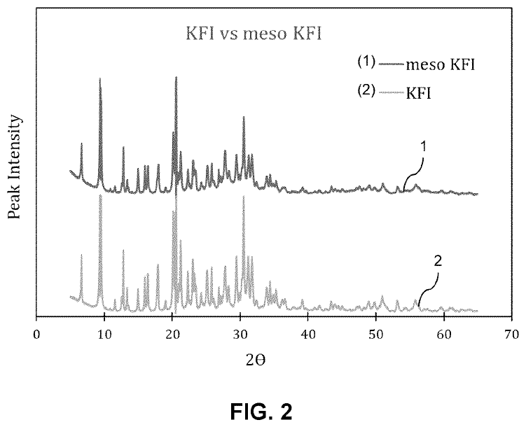

A smaller pore zeolite material of ZK-4 topology (KFI family) was synthesized in-house. The samples were obtained by hydrothermal synthesis using a templating approach at 150° C. The molar composition of the starting mixture was: 10 SiO₂: 1 Al₂O₃: 2.3 K₂O: 0.10 SrO: 1.0 (18-crown-6): 220 H₂O. A mixture of KOH (3.00 g) and Al(OH) 3 (1.56 g) with a portion of water (11.00 g) was boiled under continuous stirring until a clear solution “A” was formed and then cooled to room temperature. Sr(NO₃) 2 (0.21 g) and 18-crown-6 (2.7 g) were dissolved successively in another portion of water (8.00 g) and colloidal silica (15.00 g) was poured slowly in the thoroughly stirred solution before adding solution A. The resulting gel was then stirred for about 30 min. The pH of the starting mixture was higher than 13. The crystallization was carried out under static conditions in a polytetrafluoroethylene (PTFE) lined stainless-steel autoclave for 3, 4.5, 5 and 8 days (for different batches respectively) at 150° C. After reaction (pH~12.5-13), the solid obtained was centrifuged, washed with distilled water until the pH of the filtrate was neutral, and then dried sample at 60° C. overnight. And finally, the sample was calcined at 550° C. The synthetic procedure was based on a published report 3. Zeolites 1996, 17, 328-333 and authored by Schulz et. al. The sample was designated as KFI zeolite-B6 templated and was used as a support material for metal loading prior to applying the catalytic reaction. The X-Ray diffractogram (XRD) of the powder, typical to ZK-4 type finger print, is shown in FIG. 2. The specific surface area of the material thus obtained is recorded as 190 m²/g. The resulting material is referred to herein as the “KFI” or “synthesized KFI”.

Another facile synthesis approach adopted as per the synthesis procedure described by Kim et al. (ACS Catal. 2017, 7, 6070-6081). The KFI zeolite was synthesized via the hydrothermal conversion of a large pore zeolite in presence of Na⁺ and K⁺ ions via the inter-zeolite transformation (seeding). Commercial zeolites Y (CBV712, Si/Al₂=12) was purchased from Zeolyst. Before the synthesis, CBV712 was calcined at 550° C. for 2 h to decompose ammonium ion. In a typical synthesis (i.e., standard conditions), 1.72 g of NaNO₃, 5.11 g of KNO₃, and 4.15 g of a 1 M NaOH (Sigma-Aldrich™) solution were added to 11.1 g of deionized water in a 40 mL Teflon cup. After mixing to dissolve all salts for 1 h, 0.50 g of calcined zeolite Y with a Si/Al₂ ratio of 12 (CBV712) was added and stirred for about 1 min. The resulting mixture was transferred to a stainless-steel autoclave and kept at 140° C. for 3 days in a static oven. Then, the products were filtered, washed with deionized water, and dried at 120° C. The sample was designated as KFI zeolite-seeding B2 and were used as support material for metal loading prior to apply for catalytic reaction.

An important aspect to consider in the design of zeolite-based catalysts is the intracrystalline diffusion of gas molecules in narrow micropores, since it may restrict the performance of zeolites in adsorption and desorption processes, central to catalytic conversion. Limitations in the diffusion not only reduce the catalytic performance but also affect the selectivity and durability of the catalyst. The present example provides a strategy to tackle diffusion limitations in zeolites by incorporating different types of porosities which could potentially enhance the overall mass transport of reagents and products to and from the catalytically active sites. In particular a micro-/mesoporous material with well-defined morphology and high catalytic activity is preferable, since mesopores have a desired pore size domain for improved mass transport as well as the well-defined morphology with uniform size (spheres in micro-size range) influences the rapid adsorption and desorption of the molecules. The material was synthesized using a templating approach by using 10 g CTAB [(1-Hexadecyl) trimethyl-ammonium bromide, 98%] and mixed with 4 wt. % Ce (from CeN₃O₉·6H₂O) dissolved in deionized (DI) water, stirred it until get clear solution. Then 400 mL of 2.5 wt. % ammonia solution (pH=11.42) was added. The synthesized KFI of about 5 g was added into the mixture which was stirred for 4 h (pH=11.38). Finally, it was poured it in to the autoclave for a hydrothermal treatment at 140° C. for 24 hrs. The solution was recovered and dried at 110° C. overnight and calcined it at 550° C. for 5 hrs. The obtained sample was designated as meso-Ce—KFI (m.B6 w/Ce) and used as a support material for metal loading prior to apply for catalytic reaction. The representative powder X-Ray diffractogram (XRD), typical to ZK-4 type finger print is shown in FIG. 2 where it has been compared with the respective microporous synthesized KFI. There is a significant improvement in the specific surface area values of the material thus obtained (400 m²/g) compared to the parent material.

Another material was synthesized using a templating approach by using 10 g CTAB [(1-Hexadecyl) trimethyl-ammonium bromide, 98%] without the use of ceria. A clear solution of CTAB was mixed with 400 mL of 2.5 wt. % ammonia solution to obtain a solution (pH=11.42). The synthesized KFI of about 5 g was added into the mixture and kept it stirred for 4 h (pH=11.38). Finally, it was poured it in to the autoclave for hydrothermal treatment at 140° C. for 24 hrs. The solution was recovered and dried at 110° C. overnight and calcined it at 550° C. for 5 hrs. The sample was designated as meso-KFI (m.B6 w/o Ce) and used as support material for metal loading prior to apply for catalytic reaction.

The sodium-potassium version of the synthesized KFI material obtained was subjected to solid-solid ion exchange reaction. The as synthesized version of KFI (3.0 g) was first ground gently ground using a mortar and pestle to obtain a fine powder and recovered. Further the precursor (Fe(NO₃)₃·9H₂O, 1.0 g) was put in a large mortar and was ground with a pestle until a homogeneous powder was obtained. Both fine powders were further mixed until a homogeneous fine powder to be collected into a large crucible. The crucible was placed inside the furnace (Vulcan™ D-1750) with natural air flow. The sample was calcined under air according to the following schedule:
1) 1 hour ramp from room temperature to 120° C.
2) Dwell for 2 hours at 120° C.
3) 3.5 hours ramp from 120° C. to 550° C.
4) Dwell for 3 hours at 550° C., then hitting was stopped
5) Cool down to room temperature while keeping the sample inside the furnace (no control)
6) Total calcination time 9.5 hours

The powder thus obtained was pressed with 12 metric tons using a 1-inch die set, crushed using a mortar and pestle, then sieved to 500-710 μm before testing and was designated as sample B. The comparative chart of elemental concentrations, as determined by SEM/EDAX (scanning microscopy imaging/energy dispersive X-ray analysis) are shown in Tables 1A and 1B. The nominal iron content was 3.4 wt. % in the sample.

TABLE 1A
Chemical composition of catalyst samples
A-D synthesized and tested

| | Sample | | | | | | | |
|---|---|---|---|---|---|---|---|---|
| Sample | Name | O | Al | Si | Fe | Zn | Na | K |
| A | Fe-HKFI | 55.04 | 5.62 | 35.07 | 0.25 | — | — | — |
| | (8 times | | | | | | | |
| | Exchange) | | | | | | | |
| B | Fe/KFI | 53.16 | 6.01 | 33.22 | 2.98 | — | 0.75 | 6.42 |
| | (Solid Ion | | | | | | | |
| | Exchange) | | | | | | | |
| C | Fe/KFI | 55.67 | 7.37 | 33.66 | 2.82 | — | — | 1.31 |
| | (1-time NH4 + | | | | | | | |
| | Solid Ion | | | | | | | |
| | Exchange) | | | | | | | |
| D | Fe/KFI | 55.53 | 7.29 | 33.44 | 2.85 | — | — | 0.13 |
| | (2 times | | | | | | | |
| | NH4 + Solid | | | | | | | |
| | Ion | | | | | | | |
| | Exchange) | | | | | | | |

TABLE 1B
Chemical composition of all the tested catalyst samples

| Catalyst | | |
|---|---|---|
| Sample | Sample Composition | Comment |
| X | Fe:Zn = 1:1 (wt %) | no zeolite |
| Y | Cr—Zn—Al/Py-MOR | Pyridine -modified Mordenite |
| | (1:1) | Zeolite |
| A | Fe/KFI | KFI zeolite (8 MR) and solid ion |
| | (nominal 3.4 wt % Fe | exchange metal loading |
| | and balanced KFI | |
| | zeolite) | |
| B | Fe/KFI | KFI zeolite (8 MR) - liquid ion |
| | (nominal 3.4 wt % Fe | exchnge (4 times ammonium |
| | and balanced KFI | exchange) followed by 4 times Fe- |
| | zeolite) | nitrate salt using wet impregnation) |
| C | Fe/KFI | KFI zeolite (8 MR) - liquid ion |
| | (nominal 3.4 wt % Fe | exchange (1 time ammonium |
| | and balanced KFI | exchange) followed by Fe-loading |
| | zeolite) | by solid ion exchange using Fe- |
| | | nitrate salt) |
| D | Fe/KFI | KFI zeolite (8 MR) - liquid ion |
| | (nominal 3.4 wt % Fe | exchange (2 times ammonium |
| | and balanced KFI | exchange) followed by Fe-loading |
| | zeolite) | by solid ion exchange using Fe- |
| | | nitrate salt) |
| E | Fe—Zn / KFI | KFI zeolite (8 MR) - solid ion |
| | (nominal 3.4 wt % Fe, | exchange metal loading - impact of |
| | 0.9 wt % Zn and | additional metal loading such as Zn |
| | balanced KFI zeolite) | |
| F | Fe—Zn / KFI | KFI zeolite (8 MR) - solid ion |
| | (nominal 3.4 wt % Fe, | exchange metal loading - impact of |
| | 0.9 wt % Zn and | KFI synthesis (template vs seeding) |
| | balanced KFI zeolite) | |
| G | Fe—Zn / KFI | KFI zeolite (8 MR) - solid ion |
| | (nominal 7 wt % Fe, 0.9 | exchange metal loading - impact of |
| | wt % Zn and balanced | higher Fe concentration |
| | KFI zeolite) | |
| H | Fe—Zn / KFI | KFI zeolite (8 MR) solid ion |
| | (nominal 7 wt % Fe, 0.9 | exchange metal loading - impact of |
| | wt % Zn and balanced | higher CO2 concentration in the |
| | KFI zeolite) | feed |
| H | Fe—Zn / KFI | Mesoporous KFI zeolite (8 MR) - |
| | (nominal 7 wt % Fe, 0.9 | impact of mesoporosity with Ceria |
| | wt % Zn and balanced | |
| | KFI zeolite) | |
| I | Fe—Zn / KFI | Mesoporous KFI zeolite (8 MR) - |
| | (nominal 7 wt % Fe, 0.9 | impact of mesoporosity using w/o |
| | wt % Zn and balanced | Ceria |
| | KFI zeolite) | |

The as synthesized version of Na—K—KFI (synthesized KFI) material was converted to NH₄—KFI version through four consecutive aqueous ions exchange reactions. Prior to exchange, the KFI material was dried overnight. Using a 1-L round bottom flask (RBF) 10 g of Na—K—KFI and 500 mL of 1 (M) ammonium nitrate (NH₄NO₃) solution were mixed together in rotary evaporator and the temperature was set at 70° C. The rotavapor provided the gentle rotating that is required to initiate exchange reaction. The contents of the RBF were stirred at 70° C. for 3 hrs before cooling down to 40° C. The content was filtered using Grade 5 ashless filter paper (Buchner Funnel-Porcelain) and the filter cake was washed with 500 ml of distilled water and dried in an oven at 120° C. overnight. The procedure was repeated four times in order to complete the four exchange to remove sodium and potassium to convert the Na—K—KFI to NH₄—KFI. The sample was not calcined prior to metal loading.

The ammonium form of KFI was used as a support material to impregnate iron through an aqueous ion exchange reaction. About 4.25 gram NH₄—KFI was added to a 500 mL RBF and fitted into the rotary evaporator. The total required (Fe(NO₃)₃,9H₂O salt was estimated for 3.4% iron loading was 1.06 gram. Only one fourth (0.265 g) of this total requirement was used in step 1 by dissolving it in 212.5 mL of DI water and were added to the RBF containing NH₄—KFI and temperature was set to 70° C. The rotavapor provided the gentle rotating that is required to initiate the wet impregnation reaction. The contents of the RBF were stirred at 70° C. for 3 hrs before cooling down to 40° C. The content was filtered using Grade 5 ashless filter paper (Buchner Funnel-Porcelain) and the filter cake was washed with 500 mL of distilled water and dried in an oven at 120° C. overnight. The procedure was repeated four times in order to complete the total exchange steps to impregnate the desired loading of iron. The sample was calcined under air according to the same schedule detailed above.

The powder thus obtained was pressed with 12 metric tons using a 1-inch die set, crushed using a mortar and pestle, then sieved to 500-710 μm before testing and was designated as sample A. The comparative chart of elemental concentrations, as determined by SEM/EDAX are shown in Table 1A above. The nominal iron content was 3.4 wt. % in the sample.

The as synthesized sample of Na—K—KFI material was converted to NH₄—KFI version through one aqueous ions exchange reaction. Prior to exchange the KFI material was dried overnight. Using a 250-mL round bottom flask (RBF) 3 g of Na—K—KFI and 150 ml of 1 (M) ammonium nitrate (NH₄NO₃) solution were mixed together in rotary evaporator and the temperature was set at 70° C. The rotavapor provided the gentle rotating that is required to initiate exchange reaction. The contents of the RBF were stirred at 70° C. for 3 hrs before cooling down to 40° C. The content was filtered using Grade 5 ashless filter paper (Buchner Funnel-Porcelain) and the filter cake was washed with 250 mL of distilled water and dried in an oven at 120° C. overnight. The procedure provided a partial exchange of sodium and potassium in the as synthesized version to convert to Na—K—NH₄—KFI. Again, the sample was not calcined prior to metal loading.

The partial ammonium exchanged form of KFI was used as a support material to impregnate iron through a solid ion exchange reaction. The NH₄—KFI sample (2.7 g) was first ground gently using a mortar and pestle to obtain a fine powder and recovered. Further the precursor (Fe(NO₃)₃,9H₂O, 0.67 g) was put in a large mortar and were ground with a pestle until a homogeneous powder was obtained. Both the fine powders were further mixed until a homogeneous fine powder to be collected into a large crucible. The crucible was placed inside the furnace (Vulcan™ D-1750) with natural air flow. The sample was calcined under air according to the same schedule detailed above.

The powder thus obtained was pressed with 12 metric tons using a 1-inch die set, crushed using a mortar and pestle, then sieved to 500-710 μm before testing and was designated as sample C. The comparative chart of elemental concentrations, as determined by SEM/EDAX are shown in Table 1A. The nominal iron content was 3.4 wt. % in the sample.

The as synthesized sample of Na—K—KFI material was converted to NH₄—KFI version through two aqueous ions exchange reactions. Prior to exchange the KFI material was dried overnight. Using a 250-mL round bottom flask (RBF) 3 g of Na—K—KFI and 150 ml of 1 (M) ammonium nitrate (NH₄NO₃) solution were mixed together in rotary evaporator and the temperature was set at 70° C. The rotavapor provided the gentle rotating that is required to initiate exchange reaction. The contents of the RBF were stirred at 70° C. for 3 hrs before cooling down to 40° C. The content was filtered using Grade 5 ashless filter paper (Buchner Funnel-Porcelain) and the filter cake was washed with 250 mL of distilled water and dried in an oven at 120° C. overnight. The procedure was repeated two times in order to complete the two exchanges required to remove more sodium and potassium to convert more completely the Na—K—KFI exchange to NH₄—KFI. Again, the sample was not calcined prior to metal loading.

The improved ammonium exchanged form of KFI was used as a support material to impregnate iron through a solid ion exchange reaction. The NH₄—KFI sample (2.6 g) was first ground gently using a mortar and pestle to obtain a fine powder and recovered. Further the precursor (Fe(NO₃)₃,9H₂O, 0.65 g) was put in a large mortar and were ground with a pestle until a homogeneous powder was obtained. Both the fine powders were further mixed until a homogeneous fine powder to be collected into a large crucible. The crucible was placed inside the furnace (Vulcan™ D-1750) with natural air flow. The sample was calcined under air according to the same schedule.

The powder thus obtained was pressed with 12 metric tons using a 1-inch die set, crushed using a mortar and pestle, then sieved to 500-710 μm before testing and was designated as sample D. The comparative chart of elemental concentrations, as determined by SEM/EDAX are shown in Table 1A. The nominal iron content was 3.4 wt. % in the sample.

The sodium-potassium version of the synthesized KFI material obtained was subjected to solid-solid ion exchange reaction. The as synthesized version of KFI (2.3 g) was first ground gently using a mortar and pestle to obtain a fine powder and recovered. Further the precursors (Fe(NO₃)₃·9H₂O, 0.5 g) and (Zn(NO₃)₂·6H₂O, 0.098 g) was put in a large mortar and were ground with a pestle until a homogeneous powder was obtained. Both the fine powders (zeolites and the metal nitrates salt) were further mixed until a homogeneous fine powder to be collected into a large crucible. The crucible was placed inside the furnace (Vulcan™ D-1750) with natural air flow. The sample was calcined under air according to the same schedule as above.

The powder thus obtained was pressed with 12 metric tons using a 1-inch die set, crushed using a mortar and pestle, then sieved to 500-710 μm before testing and was designated as sample E. The nominal iron and zinc content were 3 wt. % and 1 wt. % respectively.

The sodium-potassium version of the synthesized KFI material was subjected to solid-solid ion exchange reaction. The as synthesized version of KFI (2.5 g) was first ground gently using a mortar and pestle to obtain a fine powder and recovered. Further the precursors (Fe(NO₃)₃·9H₂O, 0.55 g) and (Zn(NO₃)₂·6H₂O, 0.106 g) was put in a large mortar and were ground with a pestle until a homogeneous powder was obtained. Both the fine powders (zeolites and the metal nitrates salt) were further mixed until a homogeneous fine powder to be collected into a large crucible. The crucible was placed inside the furnace (Vulcan™ D-1750) with natural air flow. The sample was calcined under air according to the same schedule as above.

The powder thus obtained was pressed with 12 metric tons using a 1-inch die set, crushed using a mortar and pestle, then sieved to 500-710 μm before testing and was designated as sample F. The nominal iron and zinc content were 3.0 wt. % and 1 wt. % respectively.

The sodium-potassium version of the synthesized KFI material was subjected to wetness impregnation to load the metals. The as synthesized version of KFI (2.5 g) was first ground gently using a mortar and pestle to obtain a fine powder and recovered. Further the precursors (Fe(NO₃)₃·9H₂O, 1.281 g) and (Zn(NO₃) 2.6H₂O, 0.106 g) was put in a small beaker and dissolved using 3 mL of DI water to obtain a clear colored solution. The salt solution was added dropwise on the solid zeolite powder under constant stirring to obtain a slightly wet mass with homogenous distribution. The sample thus obtained was dried in an oven at 65° C. overnight. The sample was further treated at high temperature for calcination step under air according to the same schedule as above.

The powder thus obtained was pressed with 12 metric tons using a 1-inch die set, crushed using a mortar and pestle, then sieved to 500-710 μm before testing and was designated as sample G. The nominal iron and zinc content were 7 wt. % and 1 wt. % respectively.

The as synthesized version of meso-Ce—KFI (m.B6 w/Ce, 2.5 g) was first ground gently using a mortar and pestle to obtain a fine powder and recovered. Further the precursors (Fe(NO₃)₃·9H₂O, 1.281 g) and (Zn(NO₃) 2.6H₂O, 0.106 g) was put in a small beaker and dissolved using 3 mL of DI water to obtain a clear colored solution. The salt solution was added dropwise on the solid zeolite powder under constant stirring to obtain a slightly wet mass with homogenous distribution. The sample thus obtained was dried in an oven at 65° C. overnight. The sample was further treated at high temperature for calcination step under air according to the same schedule as above.

The powder thus obtained was pressed with 12 metric tons using a 1-inch die set, crushed using a mortar and pestle, then sieved to 500-710 μm before testing and was designated as sample H. The nominal iron and zinc content were 7 wt. % and 0.93 wt. % respectively.

The as synthesized version of meso-KFI (m.B6 w/o Ce, 2.4 g) was subjected wetness impregnation method to load the metals. The meso-KFI (m.B6 w/o Ce, 2.4 g) was first ground gently using a mortar and pestle to obtain a fine powder and recovered. Further the precursors (Fe(NO₃)₃·9H₂O, 1.23 g) and (Zn(NO₃)₂·6H₂O, 0.102 g) was put in a small beaker and dissolved using 3 mL of DI water to obtain a clear colored solution. The salt solution was added dropwise on the solid zeolite powder under constant stirring to obtain a slightly wet mass with homogenous distribution. The sample thus obtained was dried in an oven at 65° C. overnight. The sample was further treated at high temperature for calcination step under air according to the same schedule as above.

The powder thus obtained was pressed with 12 metric tons using a 1-inch die set, crushed using a mortar and pestle, then sieved to 500-710 μm before testing and was designated as sample I. The nominal iron and zinc content were 7 wt. % and 0.934 wt. % respectively.

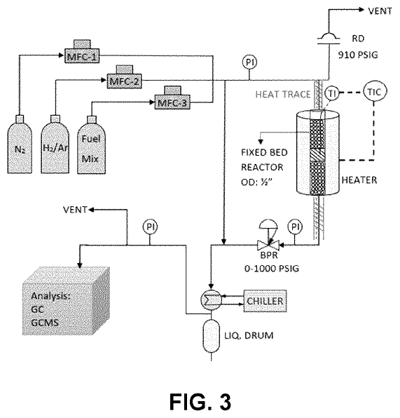

The impact of the method of metal loading (Aqueous vs. Solid Exchanges) was then evaluated. The testing apparatus is shown in FIG. 3. 1.6 g of catalysts A, B, C or D were loaded in the reactor and evaluated for syngas conversion under the following conditions: T=380° C., P=400 psi, GHSV=1032/h with a feed composition: 56% H₂, 28% CO, 10% CO₂, 6% Ar. The average conversions and overall selectivity (carbon based, mol %) over a 20-hour period are as shown in Table 2.

TABLE 2
Results for A, B, C and D.

| Metrics | A | B | C | D |
|---|---|---|---|---|
| CO conversion, % | 93 | 80 | 86 | 88 |
| Methane selectivity, % | 14.6 | 34 | 35.5 | 37.5 |
| CO₂ selectivity, % | 30 | 36 | 34 | 33 |
| C2 to C4 paraffins | 6 | 27.6 | 14 | 14 |
| selectivity, % | | | | |
| C2 to C4 olefins | 24.8 | 0.8 | 11.7 | 10.9 |
| selectivity, % | | | | |
| C5+ & Others | 24.3 | 1.5 | 4.9 | 4.6 |
| selectivity (balance), % | | | | |
| C1 to C4 hydrocarbon | 65 | 97 | 92 | 94 |
| selectivity (CO₂ free | | | | |
| basis), % | | | | |

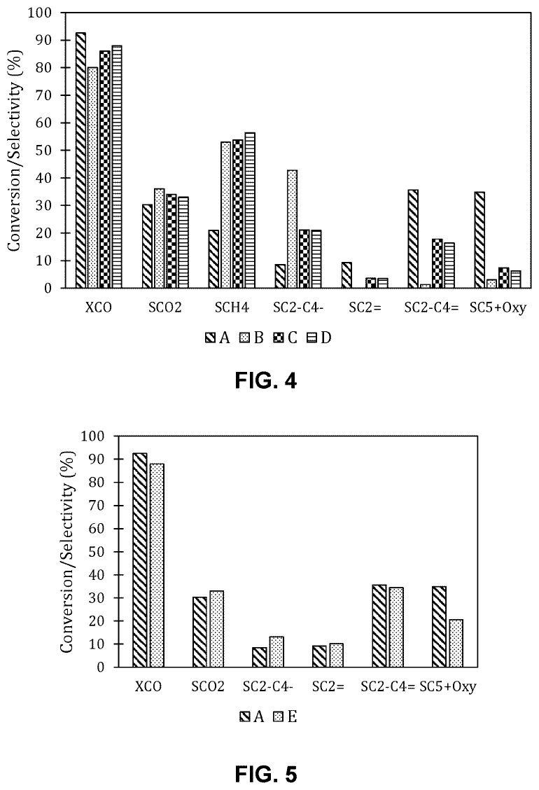

The catalysts composition as per different loading methods demonstrated a higher selectivity (from 65% to >90%) to lighter hydrocarbons (FIG. 4). The presence of potassium in the composition helped to reduce the methane selectivity significantly. Solid exchange synthesis process lowers the alkane production which is improving olefins selectivity.

The impact of Zn on Fe(1 wt. %) was then tested by providing 1.6 to 2.0 g of catalysts B and E were loaded in a reactor and evaluated for syngas conversion under the following conditions: T=380° C., P=400 psi, GHSV=1032-1500/h with a feed composition: 56% H₂, 28% CO, 10% CO₂, 6% Ar. The average conversions and overall selectivity (carbon based, mol %) over a 20-hour period are shown in Table 3.

TABLE 3
Results for the evaluation of Zn on A and E.

| Metrics | A | E |
|---|---|---|
| CO conversion, % | 93 | 88 |
| Methane selectivity, % | 14.6 | 21.2 |
| CO₂ selectivity, % | 30 | 33 |
| C2 to C4 paraffins selectivity, % | 6 | 8.7 |
| C2 to C4 olefins selectivity, % | 24.8 | 23 |
| C5+ & Others selectivity | 24.3 | 14.1 |
| (balance), % | | |
| C1 to C4 hydrocarbon selectivity | 65 | 79.4 |
| (CO₂ free basis), % | | |

As indicated, the iron catalysts with and without zinc in similar iron loading and using solid exchange method results into higher selectivity (from 65% to 80%) to lighter hydrocarbons (FIG. 5) however, with no clear effect on hydrocarbon distribution. The presence of Zn could facilitate the interaction between Zn and Fe, that could inhibit the reduction of iron oxide but enhances the CO adsorption on the iron surface, therefore making the catalyst favorable for production of long chain hydrocarbons. However, the process is restricted by high methane productivity, as well as the associated water gas shift (WGS) activity, which produces large quantities of undesired CO₂. The effect of Zn is more appreciable when present with bulk iron catalyst (X) as it helps on crystalline size of iron species, especially the crystalline size of reduced iron species, which can further impact the catalytic performance of iron catalysts for light olefins synthesis from syngas.

The impact of the Fe content loading by solid exchange (3 wt. % vs 7 wt. %) was evaluated by providing 1.6 to 2.0 g of catalysts B, E, F, or G that were loaded in a reactor and evaluated for syngas conversion under the following conditions: T=380° C., P=400 psi, GHSV=1032-1500/h with a feed composition: 56% H₂, 28% CO, 10% CO₂, 6% Ar. The average conversions and overall selectivity (carbon based, mol %) over a 20-hour period are shown in Table 4.

TABLE 4
Results for A, E, F, or G.

| Metrics | A | E | F | G |
|---|---|---|---|---|
| CO conversion, % | 93 | 88 | 76 | 86 |
| Methane selectivity, % | 14.6 | 21.2 | 26 | 31 |
| CO₂ selectivity, % | 30 | 33 | 40 | 35 |
| C2 to C4 paraffins | 6 | 8.7 | 13.9 | 15.3 |
| selectivity, % | | | | |
| C2 to C4 olefins | 24.8 | 23 | 14.2 | 12.4 |
| selectivity, % | | | | |
| C5+ & Others | 24.3 | 14.1 | 5.4 | 6.2 |
| selectivity (balance), % | | | | |
| C1 to C4 hydrocarbon | 65 | 79.4 | 90.5 | 90.4 |
| selectivity (CO₂ free | | | | |
| basis), % | | | | |

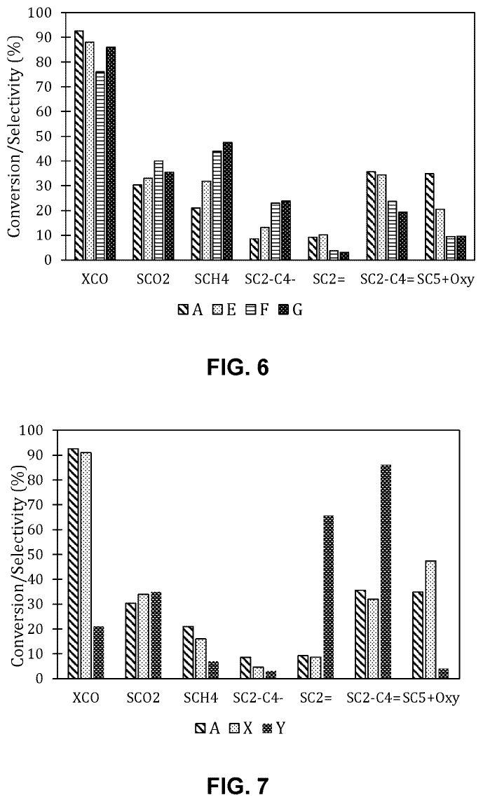

As indicated, the catalysts composition as per different loading methods demonstrated a higher selectivity (from 65% to >90%) to lighter hydrocarbons (FIG. 6). The higher loading of iron relatively results into higher CO₂ selectivity however, the higher range hydrocarbon formation is limited with higher iron loading. The CO conversion only marginally improves at higher loading. Product selectivity is also impacted as the support materials are changed.

The evaluation of the impact of zeolite presence in the catalyst formulation was performed by providing 1.6 to 2.0 g of catalysts B, X or Y that were loaded in a reactor and evaluated for syngas conversion under the following conditions: T=380° C., P=400 psi, GHSV=1032-1500/h with a feed composition: 56% H₂, 28% CO, 10% CO₂, 6% Ar. The average conversions and overall selectivity (carbon based, mol %) over a 20-hour period are shown in Table 5.

TABLE 5
Results evaluating the impact of zeolite

| Metrics | X | A | Y |
|---|---|---|---|
| CO conversion, % | 91 | 93 | 21 |
| Methane selectivity, % | 10.5 | 14.6 | 4.5 |
| CO₂ selectivity, % | 34 | 30 | 35 |
| C2 to C4 paraffins | 3.1 | 6 | 2.1 |
| selectivity, % | | | |
| C2 to C4 olefins | 21.2 | 24.8 | 55.8 |
| selectivity, % | | | |
| C5+ & Others | 31.3 | 24.3 | 2.6 |
| selectivity (balance), % | | | |
| C1 to C4 hydrocarbon | 52.6 | 65 | 96.0 |
| selectivity (CO₂ free | | | |
| basis), % | | | |

As indicated, the catalysts composition without zeolite and with zeolite have a huge impact on lighter hydrocarbon selectivity (from 52.6% to >90%) to lighter hydrocarbons (FIG. 7). The accessibility of smaller pores as presented in the zeolite through the formulations (Y and B) provide a close proximity of acidity in the zeolites (8-MR) that leads to higher selectivity to lower range hydrocarbon. The higher CO conversion at low iron loading (X vs B) also provides an opportunity to reduce water-gas-shift reaction to control CO₂ selectivity. The highest ethylene selectivity in Y also indicated that smaller pore zeolites with optimal acidity was important however, the low conversion was an issue.

The impact of higher CO₂ concentrations in the feed (10 wt. % vs 20 wt. %) was evaluated. In a two sets of experiment using the catalyst G the impact of CO₂ concentration was compared. About 2.0 g of catalysts were loaded in a reactor and evaluated for syngas conversion under the following conditions: T=380° C., P=400 psi, GHSV=1500/h with the following feed compositions:
1) 56% H₂, 28% CO, 10% CO₂, 6% Ar, and
2) 50% H₂, 24% CO, 20% CO₂, 6% Ar.

The average conversions and overall selectivity (carbon based, mol %) over a 20-hour period were recorded and presented in Table 6.

TABLE 6
Results of G with 10% or 20% CO₂ in the feed

| | G (with 10% | G (with 20% |
|---|---|---|
| Metrics | CO₂ in feed) | CO₂ in feed) |
| CO conversion, % | 86 | 82 |
| Methane selectivity, % | 31 | 37 |
| CO₂ selectivity, % | 35 | 20 |
| C2 to C4 paraffins selectivity, % | 15.3 | 18.6 |
| C2 to C4 olefins selectivity, % | 12.4 | 16.5 |
| C5+ & Others selectivity | 6.2 | 7.6 |
| (balance), % | | |
| C1 to C4 hydrocarbon selectivity | 90.4 | 91 |
| (CO₂ free basis), % | | |

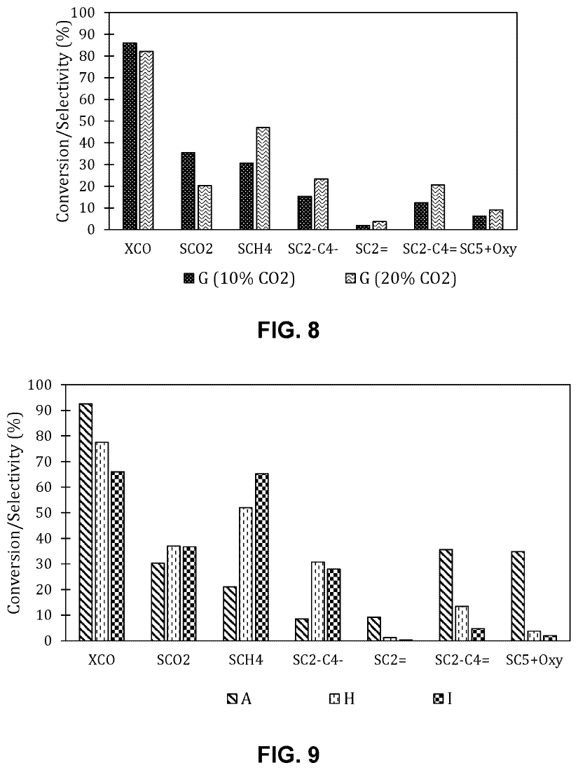

As indicated, the catalyst compositions as per different CO₂ concentration in the feed demonstrated a lower selectivity to CO₂ with almost similar (>90%) selectivity to lighter hydrocarbons (FIG. 8). The higher CO₂ in the feed also resulted into higher selectivity to lower range hydrocarbons (C2-C4). However, the methane formation was relatively higher. Higher range of hydrocarbon formation was limited by a higher of iron loading. The CO conversion had no perceptible difference, indicating that having the optimum CO₂ in the feed is beneficial for direct conversion of syngas to lower range hydrocarbon.

The impact of mesoporosity (with and without ceria) was evaluated by providing 2.0 g of catalysts B, H or I that were loaded in a reactor and evaluated for syngas conversion under the following conditions: T=380° C., P=400 psi, GHSV=1500/h with a feed composition: 56% H₂, 28% CO, 10% CO₂, 6% Ar. The average conversions and overall selectivity (carbon based, mol %) over a 20-hour period are presented in Table 7.

TABLE 7
Mesoporosity evaluation results

| Metrics | A | H | I |
|---|---|---|---|
| CO conversion, % | 93 | 78 | 66 |
| Methane selectivity, % | 14.6 | 32.8 | 41.3 |
| CO₂ selectivity, % | 30 | 37 | 37 |
| C2 to C4 paraffins | 6 | 19.4 | 17.7 |
| selectivity, % | | | |
| C2 to C4 olefins selectivity, % | 24.8 | 8.5 | 3.0 |
| C5+ & Others selectivity | 24.3 | 2.3 | 1.3 |
| (balance), % | | | |
| C1 to C4 hydrocarbon | 65 | 96.3 | 98 |
| selectivity (CO₂ free basis), % | | | |

As indicated, the catalysts composition when modified with ceria demonstrated a lower selectivity to C₅⁺ and higher methane selectivity however with very improved lower paraffins selectivity. Overall, the selectivity to lighter hydrocarbon was improved to more than 95% compared to unmodified zeolite (FIG. 9). The mesoporosity could provide the efficient mass transfer for larger molecule as well as the combined advantages of microporous zeolites (e.g., strong acid catalytic functions, shape-selective features and high stability at high temperatures owing to the crystalline structure) and mesoporous materials (e.g., efficient mass transportation for larger molecules and acid sites). The modification as demonstrated herein also improved the stability by suppressing the carbon deposition as well expected offer an opportunity to enhance the lighter hydrocarbon (C1 to C4) selectivity in syngas conversion.

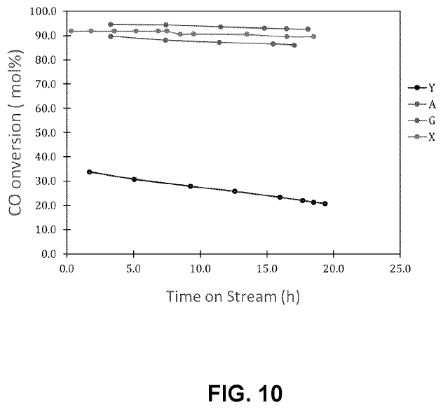

The comparative performance of the preferred catalysts that provide a direct route to light range hydrocarbon (C1 to C4) is presented in Table 8. The catalysts of the present disclosure produced a product profile without significant increase of C5⁺ production. Preferably, the catalysts produce C1 to C4 hydrocarbon range yields between 35 to 42 mol % much higher than ox-zeo (Y) catalyst. Another advantage of the present catalysts is their stability during the time-on-stream (TOS) to provide a coke-free performance (FIG. 10). The ox-zeo material is fraught with deactivation and very low conversion that does not make it a suitable catalyst for light C2-C4 hydrocarbon production by breaking the selectivity limitation determined by the ASF distribution. Further, by selecting the appropriate topology, acidity and mesoporosity of materials as well as the metal nanoparticles and support, the product selectivity profile could be tunable and improvised in the range of hydrocarbon of choice.

TABLE 8
Comparison data among the representative catalysts tested (CO₂ free basis)

| | | Selectivity | | | | Selectivity | Yields to C2 |
|---|---|---|---|---|---|---|---|
| | Single | to | Yield to | Selectivity | Yields to | to C2 to C4 | to C4 |
| | pass CO | ethylene | ethylene | to C2-C4 | C2-C4 | hydrocarbon | hydrocarbon |
| | conversion, | only, | only, | olefins, | olefins, | (P + O), | (P + O), |
| Catalyst | mol % | mol % | mol % | mol % | mol % | mol % | mol % |
| X | 91 | 8.7 | 7.9 | 32.0 | 29.1 | 36.6 | 33.3 |
| Y | 20.8 | 65.6 | 13.6 | 86.0 | 17.9 | 89.1 | 18.4 |
| A | 92.6 | 9.2 | 8.5 | 35.6 | 33 | 44.2 | 40.9 |
| E | 88 | 10.2 | 8.9 | 34.4 | 30.3 | 47.6 | 41.9 |
| G | 86 | 3.1 | 2.6 | 19.2 | 16.5 | 43.0 | 36.9 |

The principles and modes of operation of this invention have been described above with reference to various exemplary and preferred embodiments. As understood by those of skill in the art, the overall invention, as defined by the claims, encompasses other preferred embodiments not specifically enumerated herein.

While the present disclosure has been described in connection with specific embodiments thereof, it will be understood that it is capable of further modifications and this application is intended to cover any variations, uses, or adaptations, including such departures from the present disclosure as come within known or customary practice within the art and as may be applied to the essential features hereinbefore set forth, and as follows in the scope of the appended claims.

1. A catalyst for the direct conversion of syngas into light olefins, the catalyst comprising a mixture of a first catalytic component and a second catalytic component, the first catalytic component comprising an oxide of a metal selected from the group consisting of copper, silver, iron, zinc, boron, magnesium, cobalt, aluminum, vanadium, nickel, yttrium, chromium, manganese, palladium, lanthanum, zirconium, and mixtures thereof, and the second component comprising a zeolite.

2. The catalyst of claim 1, wherein the oxide of the metal of the first catalytic component comprises iron and an oxide of zinc, aluminum, manganese, copper, cobalt, and/or zirconium.

3. The catalyst of claim 1, wherein the zeolite is KFI zeolite, mordenite (MOR), metal modified MOR, and metal modified KFI.

4. The catalyst of claim 3, wherein the metal modified KFI is a mesoporous KFI zeolite containing an oxide of cerium.

5. The catalyst of claim 1, wherein the first catalytic component is an iron-based catalyst that comprises zinc oxide, manganese oxide, or a combination thereof.

6. The catalyst of claim 1, wherein the zeolite is a small or medium pore KFI type material.

7. The catalyst of claim 1, comprising from 3 wt. % to 7 wt. % Fe.

8. The catalyst of claim 1, comprising
from 0.5 wt. % to 1.0 wt. % Zn; and/or
from 0.5 wt. % to 4.0 wt. % Ce.

9. (canceled)

10. The catalyst of claim 1, comprising
53 to 56 wt. % of O, 5.5 to 7.5 wt. % of Al, 33 to 33.5 wt. % of Si, and 0 to 3.5 wt. % Fe;
55.04 wt. % O, 5.62 wt. % Al, 35.07 wt. % Si and 0.25 wt. % Fe;
53.16 wt. % O, 6.01 wt. % Al, 33.22 wt. % Si, 2.98 wt. % Fe, 0.75 wt. % Na and 6.42 wt. % K;
55.67 wt. % O, 7.37 wt. % Al, 33.66 wt. % Si, 2.82 wt. % Fe, and 1.31 wt. % K; or
55.53 t. % O, 7.29 wt. % Al, 33.44 wt. % Si, 2.85 wt. % Fe, and 0.13 wt. % K.

11-18. (canceled)

19. A process for producing light olefins from a syngas mixture comprising H₂, CO and CO₂ comprising contacting the syngas with the catalyst as defined in claim 1 in a converter unit to form a product comprising C1 to C4 light olefins.

20. The process of claim 19 where the syngas is obtained from a carbonaceous material that comprises a biomass, a plastic, an organic compound, industrial wastes, recycling facilities rejects, automobile fluff, municipal solid waste, construction and demolition debris, refuse derived fuel (RDF), solid recovered fuel, used wood utility poles, wood railroad ties, wood waste recovered form forestry, tire, synthetic textile, carpet, synthetic rubber, materials of fossil fuel origin, expanded or any combination thereof.

21-23. (canceled)

24. The process of claim 19, wherein the catalyst has a CO conversion in a range from 60 mol % to 95 mol %.

25. The process of claim 19, wherein the catalyst has a CO₂ selectivity in a range of 10 mol % to 40 mol %.

26. The process of claim 19, wherein the catalyst has a CH₄ selectivity has a range of 7 mol % to 45 mol %.

27. The process of claim 19, wherein the catalyst has C₂ to C₄ olefines selectivity in a range of 10 mol % to 25 mol %.

28. The process of claim 19, wherein the catalyst has C₂ to C₄ paraffins selectivity in a range of 4 mol % to 40 mol %.

29. The process of claim 19, wherein the catalyst has a C₅⁺ selectivity in a range of 3 mol % to 25 mol %.

30. The process of claim 19, wherein the catalyst has a selectivity range of 60 mol % to 98 mol % for C1 to C4 hydrocarbons on a CO₂ free basis

31. The process of claim 19, wherein a ratio of hydrogen to carbon monoxide in the synthesis gas is from 0.5:1 to 5:1.

32. The process of claim 19, wherein the syngas is produced from a carbonaceous gas component comprises a mixture of hydrogen and carbon monoxide and carbon dioxide with CO to CO₂ ratio between 1:1 and 3:1.

33-34. (canceled)
